(19) United States
(12) Patent Application Publication
(10) Pub. No.: US 2026/0048219 A1
(43) Pub. Date: Feb. 19, 2026

(54) BITE BLOCK

(71) Applicant: Impulse Pharmaceuticals LLC, Greenwich, CT (US)

(72) Inventor: Ethan Oliver Bryson, Toms River, NJ (US)

(73) Assignee: Impulse Pharmaceuticals LLC, Greenwich, CT (US)

(21) Appl. No.: 19/296,348

(22) Filed: Aug. 11, 2025

Related U.S. Application Data
(60) Provisional application No. 63/682,417, filed on Aug. 13, 2024.

(51) Int. Cl. A61M 16/04; A61M 16/08

(52) U.S. Cl. CPC A61M 16/0493; A61M 16/0875

(57) ABSTRACT

According to some embodiments, a bite block configured to receive a tube for intubation is provided. In some embodiments, the bite block may include a connecting portion and at least two winged portions extending outwardly from the connecting portion. In some embodiments, the connecting portion may include a channel extending between a front face and a rear face of the connecting portion with the channel being configured to receive an airway tube for intubation. The at least two winged portions may be configured to contact at least some molars of the subject when the bite block is positioned in the subject's mouth, and the winged portions may be configured to space the subject's cheek away from the subject's teeth.

### RELATED APPLICATIONS

This application claims the benefit of U.S. Provisional Patent Application Ser. No. 63/682,417, filed Aug. 13, 2024, entitled “Bite Block,” which is incorporated herein by reference.

### FIELD

Disclosed embodiments are related to bite blocks configured to protect dental structures when bitten down upon, along with associated methods.

### BACKGROUND

During a variety of medical procedures, bite blocks may be positioned in a subject's mouth to space apart the subject's teeth and retain the mouth in an open position. Some medical procedures may also necessitate the use of airway management devices such as a laryngeal mask airway (LMA) and an endotracheal tube (ETT).

### SUMMARY

In some embodiments, a bite block for a subject is provided and comprises a connecting portion and at least two winged portions extending outwardly from the connecting portion. In some embodiments, the connecting portion may include a channel extending between a front face and a rear face of the connecting portion. In some embodiments, the at least two winged portions may each extend from a first end portion adjacent to the connecting portion to a second end portion having a top surface and an outer side surface. The top surface may be configured to be positioned to contact at least some molars of the subject when positioned in a mouth of the subject, while the outer side surface may be configured to space a cheek of the subject away from teeth of the subject when positioned in the mouth of the subject.

In some embodiments, a method of using a bite block for a subject is provided. The method may include passing an airway tube through a channel formed in a connecting portion of a bite block, wherein the channel may extend between a front face and a rear face of the connecting portion. The method may further include inserting the airway tube into the subject, and positioning the bite block within a mouth of the subject such that at least two winged portions extending outwardly from the connecting portion contact at least some molars of the subject and space a cheek of the subject away from the subject's teeth.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 shows a top view of a schematic of a bite block for a subject, according to some embodiments;

FIG. 2 shows the top view of FIG. 1 in combination with an airway tube, according to some embodiments;

FIG. 3 shows a side view of a bite block according to one embodiment, where a side profile of the bite block is shown to include a continuous taper;

FIG. 4 shows a side view of a bite block according to another embodiment, where a side profile of the bite block is shown to include a stepped region;

FIG. 5 shows a front view of FIG. 1, according to some embodiments; and

FIG. 6 shows a flow diagram illustrating a method of using a bite block for a subject, according to some embodiments.

### DETAILED DESCRIPTION

During a variety of medical procedures, bite blocks may be positioned in a subject's mouth to space apart the subject's teeth and retain the mouth in an open position. The inventor(s) have recognized that certain medical procedures may cause a subject to bite down in response to an external stimulus experienced during the procedure, for example, as a result of stimulation from ECT or motor evoked potentials (MEPs). The inventor(s) have appreciated that the “biting-down” effect experienced during such procedures may cause injury to the subject, such as tongue laceration, dental injury, pulmonary injury (e.g., related to obstruction of an endotracheal tube), or other injuries.

The inventor(s) have recognized benefits associated with a bite block that may be constructed and arranged to space a cheek of the subject away from the teeth of the subject when the bite block is positioned in the subject's mouth. For example, the bite block may include winged portions extending outwardly from a connecting portion of the bite block, and the winged portions may be sized and shaped to space the subject's cheek from the subject's teeth.

Some medical procedures may also necessitate the use of airway management devices such as a laryngeal mask airway (LMA) and an endotracheal tube (ETT). Such devices may utilize an airway tube that is inserted through the subject's mouth. For example, subjects who receive anesthesia, undergo orthopedic or neurosurgical spine procedures, undergo electroconvulsive therapy (ECT), or undergo other medical procedures may require an airway management device.

In view of the above, the inventor(s) have appreciated that conventional bite blocks typically either obstruct the placement of an airway tube or only provide protection to a portion of a subject's mouth (e.g., a bite block that only protects one side of the subject's mouth). Accordingly, the inventor(s) have recognized benefits associated with providing a bite block configured to receive an airway tube (e.g., an ETT or an LMA), where the bite block provides protection to a larger portion of a subject's mouth (e.g., bilateral protection to both sides of the subject's mouth).

Although the embodiments disclosed herein are primarily discussed in reference to use of an ETT with a bite block, any suitable type of airway tube may be employed for use with the bite block, including a laryngeal mask airway (LMA).

It should be appreciated that the devices disclosed herein may be used as a bite block with or without an airway management device.

In some embodiments, a bite block as disclosed herein may be constructed of a suitable material including, but not limited to: polyethylene (e.g. expanded polyethylene foam), polystyrene, polyisoprene, polychloroprene, polybutadiene, polyisobutylene, silicone, or any other suitable material as the disclosure is not so limited.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

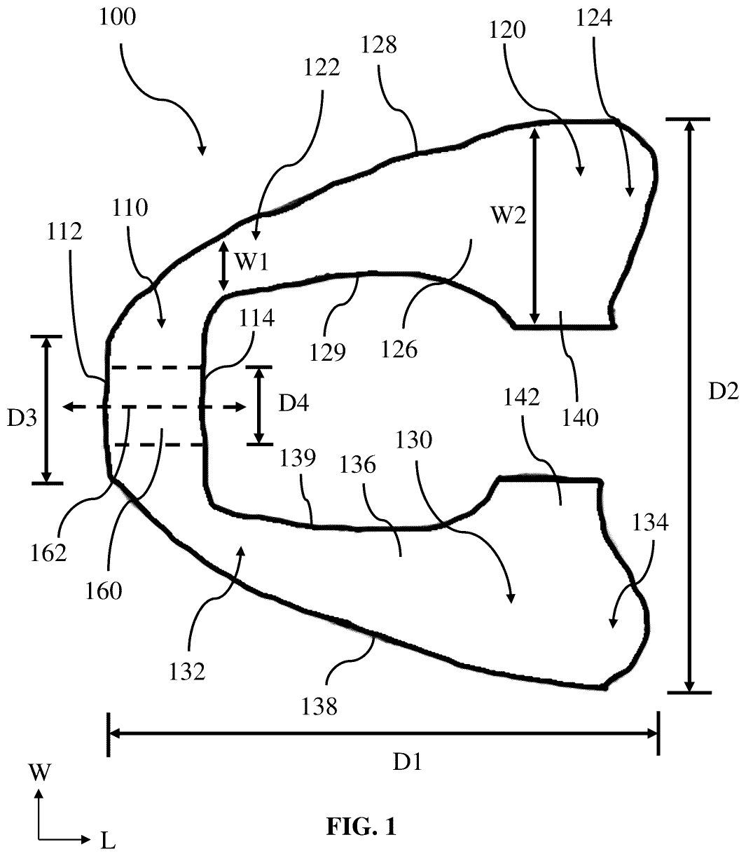

FIG. 1 shows a top schematic view of a bite block 100 configured for use with a subject. The bite block 100 may include a connecting portion 110 and at least two winged portions 120, 130 extending outwardly from the connecting portion 110. The connecting portion 110 may include a channel 160 extending between a front face 112 and a rear face 114 of the connecting portion. The channel 160 may be configured to receive a portion of an airway tube therethrough for airway management, such as for intubation (e.g., an ETT or LMA as disclosed herein). The winged portions 120, 130 may extend from respective first end portions 122, 132 adjacent the connecting portion 110 to respective second end portions 124, 134. As used herein, the winged portions 120, 130 may have respective top surfaces 126, 136 and respective outer side surfaces 128, 138. In some embodiments, the top surfaces 126, 136 may be configured to be positioned to contact at least some of the subject's teeth (e.g., the molars) when the bite block is positioned in the mouth of the subject. Additionally, at least some of the subject's teeth may contact the top surface of the connecting portion 110. In some embodiments, the outer side surfaces 128, 138 may be configured to space the subject's cheek away from the subject's teeth when the bite block is positioned in the subject's mouth. That is, the winged portions 120, 130 may be sized and shaped such that the outer side surfaces 128, 138 contact and push away the subject's cheeks. For example, the winged portions 120, 130 may have a greater width (direction W) at a location closer to the second end portions 124, 134 than the first end portions 122, 132 as shown in FIG. 1, such that the outer side surfaces 128, 138 will space away the subject's cheek at a location adjacent to the subject's molars. That is, the width between the outer side surfaces 128, 138 and respective inner side surfaces 129, 139 which oppose the outer side surfaces may increase in a direction from the first end portions 122, 132 to second end portions 124, 134. For example, as shown in FIG. 1, the width increases from width W1 at the first end portions 122, 132 to the width W2 at the second end portions 124, 134 such that width W2 is larger than W1.

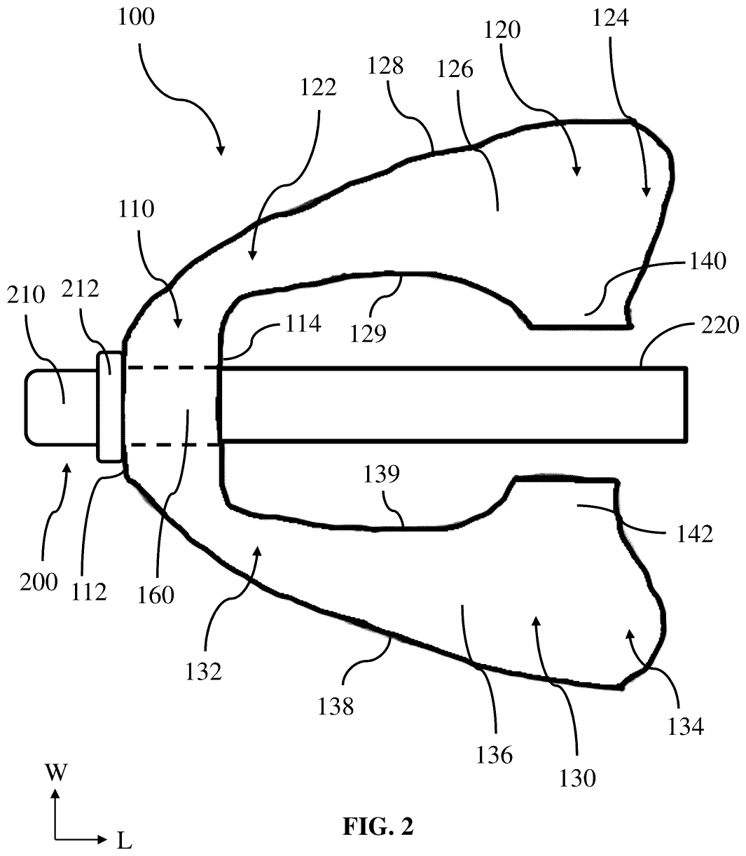

The bite block 100 may also include one or more tabs 140, 142 extending inwardly from respective winged portions 120, 130. For example, the tabs 140, 142 may extend inwardly from the second end portions 124, 134 as shown in FIG. 1. In some embodiments, the tabs may serve to increase a width W2 of the second end portions 124, 134 so that when the subject bites down on a portion of the top surfaces 126, 136 (e.g., on a portion closer to the tabs 140, 142), the outer side surfaces 128, 138 may further push away the subject's cheek from the subject's teeth. In some embodiments, the tabs may be configured to limit movement of an airway tube portion 220 (shown in FIG. 2) when the tube is received within the channel 160 of the bite block. That is, the tube may extend through channel 160 and through a gap formed between tabs 140, 142, and the tabs may extend inwardly such that lateral movement of the tube within the gap is limited. Such a configuration may help to guide the airway tube into a proper location of the subject.

As shown in FIG. 1, the bite block 100 may have a variety of dimensional parameters D1, D2, D3, and D4 such that the bite block accommodates the size and shape of the subject's mouth and the airway tube to be received within the bite block.

As used herein, “D1” may refer to a maximum longitudinal (i.e., length, see direction L) dimension of the bite block while “D2” may refer to a maximum transverse (i.e., width) dimension of the bite block. In some embodiments, the length dimension D1 may be perpendicular to the width dimension D2 as shown in FIG. 1. In some embodiments, the length dimension D1 may be parallel to a longitudinal axis 162 of the channel 160 while the width dimension D2 may be perpendicular to the longitudinal axis of the channel 160. In some embodiments, a suitable length dimension D1 of the bite block may be greater than or equal to 5 cm, 6 cm, 7 cm, 8 cm, 9 cm, 10 cm, 11 cm, 12 cm, or greater. Likewise, in some embodiments, a suitable length dimension D1 may be less than or equal to 15 cm, 14 cm, 13 cm, 12 cm, 11 cm, 10 cm, 9 cm, 8 cm, or less. Combinations of the above-referenced ranges are also possible. For example, in some embodiments, length dimension D1 of the bite block may be between or equal to 5 to 15 cm, 6 to 14 cm, 7 to 13 cm, 8 to 12 cm, 9 to 11 cm, or any other suitable range.

In some embodiments, a suitable width dimension D2 of the bite block may be greater than or equal to 5 cm, 6 cm, 7 cm, 8 cm, 8.5 cm, 9 cm, 9.5 cm, 10 cm, 10.5 cm, 11 cm, 12 cm, or greater. Likewise, in some embodiments, a suitable width dimension D2 may be less than or equal to 15 cm, 14 cm, 13 cm, 12 cm, 11 cm, 10 cm, 9 cm, 8 cm, or less. In some embodiments, a suitable width dimension D2 may be between or equal to 5 to 15 cm, 6 to 14 cm, 7 to 13 cm, 8 to 12 cm, 9 to 11 cm, or any other suitable range.

As used herein, “D3” may refer to a width dimension of the front face of the connecting portion 110. In some embodiments, a suitable width dimension D3 of the connecting portion may be greater than or equal to 1 cm, 2 cm, 3 cm, 4 cm, 5 cm, or greater. Likewise, in some embodiments, a suitable width dimension D3 may be less than or equal to 8 cm, 7 cm, 6 cm, 5 cm, 4 cm, 3 cm, or less. In some embodiments, a suitable width dimension D3 may be between or equal to 1 to 8 cm, 1 to 7 cm, 1 to 6 cm, 1 to 5 cm, 2 to 4 cm, or any other suitable range.

As used herein “D4” may refer to a width dimension of the channel 160. In some embodiments, a suitable width dimension D4 may be greater than or equal to 5 mm, 10 mm, 15 mm, 20 mm, or greater. Likewise, in some embodiments, a suitable width dimension D4 may be less than or equal to 25 mm, 20 mm, 15 mm, or less. While such ranges of dimensional parameters are disclosed above, it should be understood that dimensional parameters both greater and less than the foregoing are also contemplated. In some embodiments, a suitable width dimension D4 may be between or equal to 5 to 25 mm, 10 to 20 mm, 5 to 15 mm, 15 to 25 mm, or any other suitable range.

FIG. 2 shows the top view of the bite block 100 of FIG. 1 in combination with an airway tube 200 which may, in some embodiments, be used for intubation. The airway tube 200 may be of any suitable type as disclosed herein, including an endotracheal tube (ETT) or a laryngeal mask airway (LMA). In some embodiments, the airway tube 200 may be configured to be positioned through the channel 160, extend past the bite block (e.g., through the gap between tabs 140 and 142), and extend into the subject's trachea for intubation. The presence of such an airway tube may keep the airway open in order to give oxygen, medicine, and/or anesthesia to the subject. The inventor(s) have appreciated that by passing the airway tube 200 through a channel 160 extending through the connecting portion 110, a position of the airway tube may be stabilized and the airway tube may be protected from damage resulting from the subject biting down during a medical procedure (e.g., in response to an ECT procedure).

The airway tube 200 may be engaged with the bite block in any suitable fashion. For example, the airway tube 200 may include a first portion 210 configured to protrude from the connecting portion 110 and a retaining element 212 configured to abut the connecting portion 110 to limit a depth of insertion of the airway tube through the channel 160. That is, the retaining element 212 may have a greater width and/or height than the channel 160 such that the first portion 210 does not extend into the channel 160. The airway tube 200 may also include a second portion 220 which extends through the channel 160 and past the bite block for intubating the subject. The first and second portions 210, 220 may be of any suitable length to facilitate intubation of a subject. For example, the first portion 210 may be of a suitable length to connect to an air delivery device such as a squeezable bag to deliver air or a ventilator. Likewise, the second portion 220 may be of a suitable length to extend between the vocal cords of the subject and through the trachea. The airway tube may also be constructed of any suitable material including, but not limited to polyvinyl chloride, silicone rubber, latex rubber, metals, or any other suitable materials.

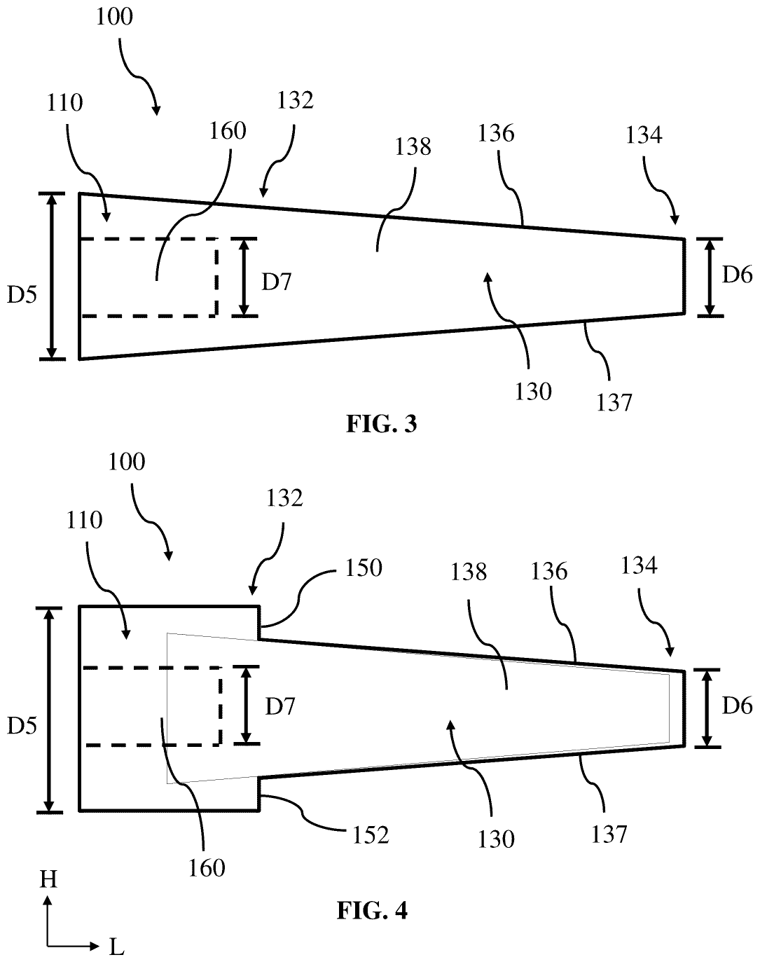

FIGS. 3-4 show embodiments of a side view of the bite block 100, where a side profile of the bite block is tapered. That is, the bite block may have a top side (defined by top surface 136) and a bottom side 137 opposing the top side with a height direction (direction H) being defined as the distance between the top and bottom sides, and the bite block may be tapered such that the height D5 of the connecting portion 110 is greater than a height D6 of the second end portion 134. It should be understood that although only a right side profile of the bite block 100 is shown in FIGS. 3-4, that the tapered profile and embodiments disclosed herein may also be applied to the left side profile including winged portion 120.

FIG. 3 shows an embodiment where the bite block is tapered in a continuous fashion from the front face of the connecting portion 110 to the second end portion 134. Accordingly, a height dimension of the front face of the connecting portion “D5” may be greater than a height dimension of the second end portion “D6”. Although FIG. 3 shows a continuously decreasing taper from connecting portion 110 to second end portion 134, in some embodiments the bite block may include an inverse taper such that at least a portion of the bite block located closer to the second end portion 134 is of a greater height than at least a portion of the bite block located closer to connecting portion 110.

FIG. 4 shows an embodiment where the bite block is tapered and includes one or more stepped regions 150, 152. The stepped regions 150, 152 may be formed along any suitable length (direction L) of the bite block such as at the first end portion 132, as shown in FIG. 4. Although FIG. 4 shows two stepped regions 150, 152, it should be understood that in some embodiments only one stepped region may be included (e.g., on the top side or bottom side of the bite block). Alternatively, in some embodiments, more than two stepped regions may be formed along the length of the bite block. For example, a first set of stepped regions 150, 152 may be formed at first end portion 132 and then a second set of one or more stepped regions may be formed at a location between first end portion 132 and second end portion 134.

The stepped regions 150, 152 may be of any suitable height as the disclosure is not so limited. In some embodiments, a suitable height of the stepped regions 150, 152 may be greater than or equal to 0.5 mm, 1 mm, 1.5 mm, 2 mm, 2.5 mm or greater. Likewise, in some embodiments, a suitable height of the stepped regions 150, 152 may be less than or equal to 5 mm, 4.5 mm, 4 mm, 3.5 mm, 3 mm, 2.5 mm, 2 mm, 1.5 mm, or less. Although FIG. 4 shows a stepped decrease in height from the connecting portion 110 to the winged portion 130, in some embodiments, the stepped regions may be constructed and arranged as a stepped increase in height from the connecting portion 110 to the winged portion 130 as the disclosure is not so limited.

In some embodiments, a suitable height dimension D5 may be greater than or equal to 10 mm, 11 mm, 12 mm, 13 mm, 14 mm, 15 mm, 16 mm, 17 mm, 18 mm, 19 mm, 20 mm, or greater. Likewise, in some embodiments, a suitable height dimension D5 may be less than or equal to 30 mm, 29 mm, 28 mm, 27 mm, 26 mm, 25 mm, 24 mm, 23 mm, 22 mm, 21 mm, 20 mm, 19 mm, 18 mm, 17 mm, 16 mm, 15 mm, or less. In some embodiments, a suitable height dimension D5 may be between or equal to 10 and 30 mm, 14 and 29 mm, 15 and 25 mm, 16 and 24 mm, 17 and 23 mm, 18 and 22 mm, 19 and 21 mm, or any other suitable range.

In some embodiments, a suitable height dimension D6 may be greater than or equal to 3 mm, 4 mm, 5 mm, 6 mm, 7 mm, 8 mm, 9 mm, 10 mm, 11 mm, 12 mm, 13 mm, 14 mm, 15 mm, or greater. Likewise, in some embodiments, a suitable height dimension D6 may be less than or equal to 20 mm, 19 mm, 18 mm, 17 mm, 16 mm, 15 mm, 14 mm, 13 mm, 12 mm, 11 mm, 10 mm, or less. In some embodiments, a suitable height dimension D6 may be between or equal to 3 and 20 mm, 4 and 16 mm, 5 and 15 mm, 6 and 14 mm, 7 and 13 mm, 8 and 12 mm, 9 and 11 mm, or any other suitable range.

As used herein, “D7” may refer to a height dimension of the channel 160 as shown in FIGS. 3-4. In some embodiments, a suitable channel height dimension D7 may be greater than or equal to 5 mm, 10 mm, 15 mm, 20 mm, or greater. Likewise, in some embodiments, a suitable height dimension D7 may be less than or equal to 25 mm, 20 mm, 15 mm, or less. In some embodiments, a suitable height dimension D7 may be between or equal to 5 and 25 mm, 6 and 24 mm, 7 and 23 mm, 8 and 22 mm, 9 and 21 mm, 10 and 20 mm, 11 and 19 mm, 12 and 18 mm, 13 and 17 mm, 14 and 16 mm, or any other suitable range. In some embodiments, width dimension D4 and height dimension D7 of the channel 160 may be sized and shaped according to the dimensional parameters of the airway tube to be received within the channel 160. That is, the channel 160 may be of a complementary size and/or shape to the airway tube 200 shown in FIG. 2 to stabilize a position of the airway tube received in the channel.

As discussed above, suitable ranges of dimensional parameters may be formed from the foregoing. For example, for a given bite block, length dimension D1 may be greater than or equal to 9 cm, width dimension D2 may be greater than or equal to 9.5 cm, width dimension D3 may be greater than or equal to 2 cm, width dimension D4 may be between or equal to 10 and 20 mm, height dimension D5 may be between or equal to 15 mm and 25 mm, height dimension D6 may be between or equal to 5 mm and 15 mm, and height dimension D7 may be between or equal to 10 mm and 20 mm. While such ranges of dimensional parameters are disclosed above, it should be understood that dimensional parameters both greater and less than the foregoing are also contemplated.

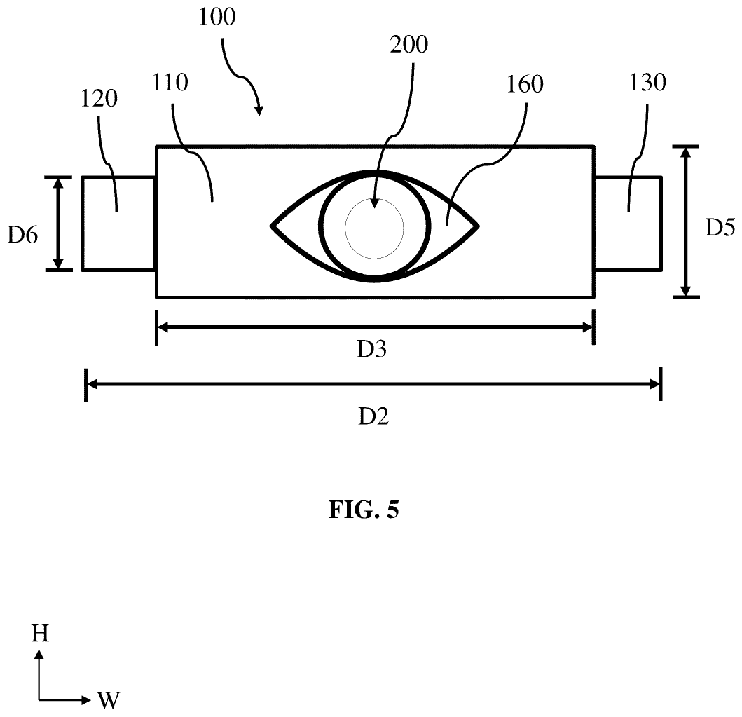

FIG. 5 shows a front view of the bite block 100 of FIG. 1 in combination with an airway tube 200. The channel 160 may be formed to have any suitable cross-sectional shape including, but not limited to, an ellipse, a circle, a square, a rectangle, an eye-shape (i.e., an ellipse with opposing pointed ends as shown in FIG. 5), or any other suitable cross-sectional shape. As discussed above, the channel 160 may be sized and shaped to receive and stabilize the position of a corresponding airway tube 200 within the channel. Although the connecting portion 110 is shown in FIG. 5 to have a continuous height dimension D5 along the front face, in some embodiments the height dimension D5 may decrease in an outward direction (i.e., in a direction extending away from channel 160 towards each of winged portions 120 and 130).

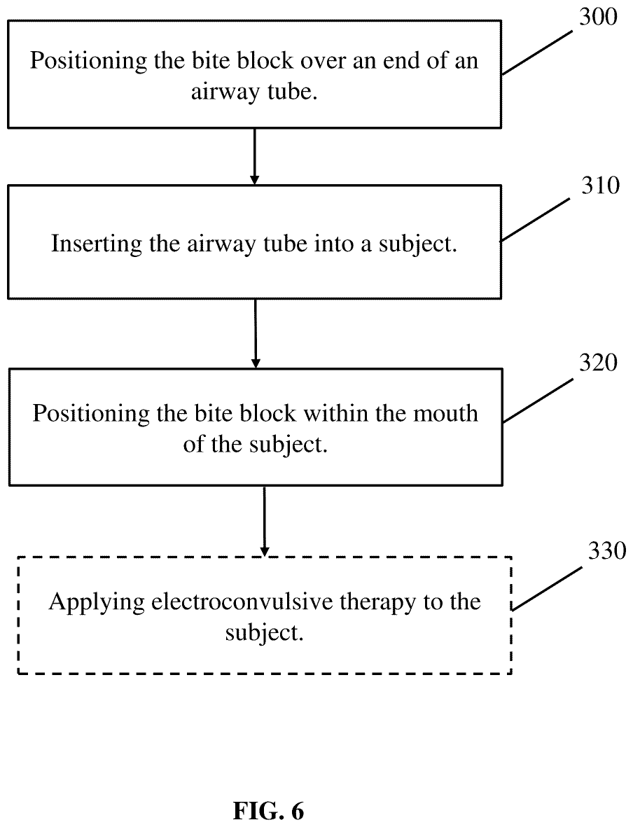

FIG. 6 shows a schematic flow diagram illustrating a method of using a bite block for a subject, according to some embodiments. In step 300, an airway tube may be passed through a channel of the bite block. As disclosed herein, the channel may extend between a front face and a rear face of a connecting portion of the bite block. In step 310, the airway tube may be inserted into the subject. For example, the airway tube may be inserted between the vocal cords of the subject and through the subject's trachea to form the airway. In step 320, the bite block may be positioned within the subject's mouth such that at least two winged portions extending outwardly from the connecting portion of the bite block contact at least some of the molars of the subject and space the subject's cheek away from the subject's teeth. In some embodiments, positioning the bite block within the subject's mouth may also include positioning the connecting portion of the bite block such that the connecting portion contacts at least one incisor of the subject.

Although FIG. 6 illustrates the step of inserting the airway tube into the subject after passing the airway tube through the channel of the bite block, the airway tube may instead be inserted into the subject before the step of passing the airway tube through the channel of the bite block as the disclosure is not so limited.

Optionally, the method may further include a step 330 of applying electroconvulsive therapy to the subject while the bite block is positioned in the subject's mouth. In some such embodiments, the bite block may be configured to protect the subject's cheeks, mouth, and/or teeth as the subject spasmodically bites down on the bite block and releases in response to the electroconvulsive therapy stimulation.

In some embodiments, a bite block is positioned in a subject's mouth without the use of an airway tube.

The embodiments described herein may be embodied as a method. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A bite block for a subject, the bite block comprising:
a connecting portion including a channel extending between a front face and a rear face of the connecting portion; and
at least two winged portions extending outwardly from the connecting portion, wherein the at least two winged portions each extend from a first end portion adjacent to the connecting portion to a second end portion having a top surface and an outer side surface, the top surface being configured to be positioned to contact at least some molars of the subject when positioned in a mouth of the subject, and the side surface being configured to space a cheek of the subject away from teeth of the subject when positioned in the mouth of the subject.

2. The bite block of claim 1, wherein a longitudinal axis of the channel runs along a length direction of the bite block, and the bite block has a width direction perpendicular to the length direction,
and wherein each of the at least two winged portions includes an inner side surface opposing the outer side surface, and wherein a width measured from the inner side surface to the outer side surface along the width direction increases from the first end portion to the second end portion.

3. The bite block of claim 1, in combination with an airway tube, wherein the airway tube is configured to be positioned through the channel.

4. The bite block of claim 1, wherein a longitudinal axis of the channel runs along a length direction of the bite block, and wherein a maximum length of the bite block measured along the length direction is greater than or equal to 9 centimeters.

5. The bite block of claim 1, wherein a longitudinal axis of the channel runs along a length direction of the bite block, and the bite block has a width direction perpendicular to the length direction, and wherein a maximum width of the bite block measured along the width direction is greater than or equal to 9.5 centimeters.

6. The bite block of claim 1, wherein a width of the front face of the connecting portion is greater than or equal to 2 centimeters, and wherein a width of the bite block at a rear side of the bite block adjacent the second end portions is greater than or equal to 5 centimeters.

7. The bite block of claim 1, wherein the bite block comprises polystyrene.

8. The bite block of claim 1, wherein the bite block consists essentially of polystyrene.

9. The bite block of claim 1, wherein the bite block is constructed of a material selected from a group of polystyrene, polyisoprene, polychloroprene, polybutadiene, polyisobutylene, or silicone.

10. The bite block of claim 1, wherein each of the second end portions includes a tab extending inwardly toward one another and spaced from one another by a gap.

11. The bite block of claim 1, wherein the bite block has a top side and a bottom side, the top surface of each of the second end portions being part of the top side, and a height direction of the bite block extends from the top side to the bottom side,
wherein the bite block is tapered such that a height of the front face of the connecting portion measured along the height direction is greater than a height of each of the second end portions of the at least two winged portions measured along the height direction.

12. The bite block of claim 11, wherein the taper of the bite block is a continuous taper.

13. The bite block of claim 11, wherein the taper of the bite block includes a stepped region.

14. The bite block of claim 11, wherein the height of the front face is between or equal to 15 and 25 mm, and wherein the height of the second end portions is between or equal to 5 and 15 mm.

15. The bite block of claim 1, wherein a height of the channel is between or equal to 10 and 20 mm.

16. The bite block of claim 1, wherein a cross-sectional shape of the channel is selected from a group of elliptical, spherical, and an ellipse with opposing pointed ends.

17. A method of using a bite block for a subject, the method comprising:
passing an airway tube through a channel formed in a connecting portion of the bite block, wherein the channel extends between a front face and a rear face of the connecting portion;
inserting the airway tube into the subject;
positioning the bite block within a mouth of the subject such that at least two winged portions extending outwardly from the connecting portion contact at least some molars of the subject and space a cheek of the subject away from the teeth.

18. The method of claim 17, wherein passing the airway tube through the channel occurs before inserting the airway tube into the subject.

19. The method of claim 17, wherein passing the airway tube through the channel occurs after inserting the airway tube into the subject.

20. The method of claim 17, wherein positioning the bite block within the mouth of the subject includes positioning the connecting portion such that the connecting portion contacts at least one incisor of the subject.

21. The method of claim 17, wherein a longitudinal axis of the channel runs along a length direction of the bite block, and the bite block has a width direction perpendicular to the length direction,
and wherein each of the at least two winged portions includes an inner side surface and an opposing outer side surface, the outer side surface configured to space the cheek of the subject away from the teeth,
and wherein a width of each of the winged portions measured from the inner side surface to the outer side surface along the width direction increases from a first end portion adjacent to the connecting portion to an opposing second end portion that contacts molars of the subject.

22. The method of claim 17, further comprising applying electroconvulsive therapy to the subject while the bite block is positioned in the subject's mouth, wherein the bite block protects the subject's cheeks, mouth and teeth as the subject spasmodically bites down on the bite block and releases.
